(19) United States
(12) Patent Application Publication
(10) Pub. No.: US 2022/0169961 A1
(43) Pub. Date: Jun. 2, 2022

(54) Aquaculture Bioreactor

(71) Applicant: Algae to Omega, LLC, Boca Raton, FL (US)

(72) Inventor: Geronimos DIMITRELOS, Plantation, FL (US)

(73) Assignee: Algae to Omega, LLC, Boca Raton, FL (US)

(21) Appl. No.: 17/108,745

(22) Filed: Dec. 1, 2020

(51) Int. Cl. C12M 1/00; C12N 1/12; C12M 1/12

(52) U.S. Cl. CPC C12M 21/02; C12M 29/18; C12M 23/06; C12N 1/12

(57) ABSTRACT

A gliding wave reactor includes a base, a tank, a suspension bracket, and an actuator. The tank is configured to hold a liquid. The suspension bracket is configured to suspend the tank over the base. The actuator is fixed to the base and configured to move the tank to create an environment in the liquid for cultivating marine life.

### FIELD

The present technology relates to a gliding wave reactor and, more specifically, a gliding wave reactor for efficiently growing aquatic life.

### BACKGROUND

This section provides background information related to the present technology which is not necessarily prior art.

Algae are a group of diverse organisms including microalgae and macroalgae, which resemble plants but lack true leaves, stems, roots and vascular tissue/systems. They are mainly characterized by their ability to carry out photosynthesis to provide all or a part of the carbon they require for growth (being primarily phototrophic, although some algae are capable of heterotrophic or mixotrophic growth based on their evolutionary mechanisms available). Algae can evolve with changes in environments and environmental conditions.

Since algae take up carbon dioxide in the process of photosynthesis, they are implicated in carbon sequestration, and can be a renewable carbon source for a variety of foodstuffs, organic compounds, and other carbon-based materials. For example, algae may be used as feedstock for animals such as fish, chickens, and cattle. They may also be a source of naturally-derived antioxidants and other materials useful in cosmetic, nutraceutical and pharmaceutical products. They are most notably known as a biofuel replacement for oil while entrenching themselves in the solar, battery and hydrogen cell industries. Among the most commercially used microalgae are Chlorella and Spirulina. In addition, Dunaliella, Haematococcus, Crypthecodinium, Schizochytrium, Scenedesmus, Aphanizomenon, Arthrospira, Odontella, Isochrysis, Nannochloropsis, Tetraselmis, Phaeodactylum, and Porphyridium are gaining acceptance in animal feed, functional food, pharmaceutical, cosmeceutical, nutraceutical, and industrial material products.

Microalgae, in particular, are amenable to commercial production, such as using photobioreactors in dedicated facilities. However, they are notoriously challenging to grow in facilities on an industrial scale because their growth depends on precision in providing the proper amount and balance of nutrients, carbon sources, light, and temperature. Human intervention is required during cultivation to ensure that the algae do not “crash” during their initial growth phase or while their growth is reaching the exponential stage. Especially at a scale necessary for feeding the world's supply of fish and other animals, algae cultivation remains a challenge. Moreover, while equipment and processes for growing algae are known in the art, such processes may not be economically feasible, requiring significant time for growth and using significant space. Accordingly, there is a need for improved methods of algae production, particularly for commercial scale production of algae for use in producing food and other products.

### SUMMARY

This section provides a general summary of the disclosure, and is not a comprehensive disclosure of its full scope or all of its features.

The present technology provides equipment, systems and methods for growing aquatic organisms, such as algae. In various embodiments, the present technology provides a vessel (e.g., a “reactor” or “gliding wave reactor”) operable to contain an aqueous media for the growth of organisms. An example gliding wave reactor according to the present technology includes a base, a tank, a suspension bracket, and an actuator. The tank is configured to hold a liquid. The suspension bracket is configured to suspend the tank over the base. The actuator is fixed to the base and configured to move the tank to create an environment in the liquid for cultivating marine life.

In an example embodiment, a tank cradle may be attached to the suspension bracket and may define a cavity for supporting the tank.

In an example embodiment, the suspension bracket may include a vertical post and a swing arm. The swing arm may be rotatably attached to the vertical post. The vertical post may be fixed to the base. The swing arm may be rotatably attached to the tank cradle.

In an example embodiment, the base and the tank cradle may be modular assemblies.

In an example embodiment, the suspension bracket may be one of a pair of suspension brackets, where the pair of suspension brackets may be disposed on either an end of the base or a side of the base for suspending the tank on either an end of the tank or a side of the tank, respectively. For example, when the pair of suspension brackets are positioned on opposing ends of the end of the base or the side of the base, the actuator may move the tank in an inverse rocking motion. As a further example, when the pair of suspension brackets are positioned spaced from opposing ends of the end of the base or the side of the base, the actuator may move the tank in a gliding motion. Also, for example, when each of the pair of suspension brackets are positioned closer to a center of the end of the base or the side of the base than an end of the end of the base or the side of the base, the actuator may move the tank in a rocking motion.

In an example embodiment, the actuator may be a linear actuator including a motor, a rod, and a housing. The rod of the actuator may be fixed to a single point on the tank.

In an example embodiment, an attachment may be fixed to a longitudinal arm supported by the suspension bracket and extending over an open top of the tank. For example, the attachment may be a light, a heat lamp, a partition, a camera, a sensor, an automatic feeder, or a combination thereof.

In an example embodiment, a controller may control the actuator.

An example embodiment of an algae-cultivating apparatus according to the present technology includes a tank, a suspension bracket, an actuator, and a controller. The tank is configured to hold a liquid. The suspension bracket is configured to suspend the tank. The actuator is fixed to the tank and configured to move the tank to create an environment in the liquid for cultivating algae. The controller is configured to control actuation of the actuator and movement of the tank.

In an example embodiment, the algae-cultivating apparatus may additionally include a base. The suspension bracket may be fixed to the base.

In an example embodiment, a tank cradle may be attached to the suspension bracket and may house the tank.

In an example embodiment, the suspension bracket may include a vertical post and a swing arm. The swing arm may be rotatably attached to the vertical post, the vertical post may be fixed to the base, and the swing arm may be rotatably attached to the tank cradle.

In an example embodiment, the base and the tank cradle may be modular assemblies.

In an example embodiment, the suspension bracket may be one of a pair of suspension brackets. The pair of suspension brackets may suspend the tank on either an end of the tank or a side of the tank.

In an example embodiment, the actuator may be a linear actuator including a motor, a rod, and a housing.

Further areas of applicability will become apparent from the description provided herein. The description and specific examples in this summary are intended for purposes of illustration only and are not intended to limit the scope of the present technology.

### DRAWINGS

FIG. 1 is a perspective view of an example embodiment of a gliding wave reactor assembly.

FIG. 2 is an exploded view of an example embodiment of the gliding wave reactor assembly illustrated in FIG. 1.

FIG. 3A is an end view of the example embodiment of the gliding wave reactor assembly illustrated in FIG. 1 moving in an inverse rock movement.

FIG. 3B is an end view of an example embodiment of the gliding wave reactor assembly illustrated in FIG. 1 moving in a gliding movement.

FIG. 3C is an end view of an example embodiment of the gliding wave reactor assembly illustrated in FIG. 1 moving in a rock movement.

FIG. 4 is a perspective view of a second configuration of the example embodiment of the gliding wave reactor assembly illustrated in FIG. 1.

FIG. 5 is a perspective view of the example embodiment of the gliding wave reactor assembly illustrated in FIG. 1.

Corresponding reference numerals indicate corresponding parts throughout the several views of the drawings.

It should be noted that the figures set forth herein are intended to exemplify the general characteristics of devices, systems, components, materials and methods among those of the present technology, for the purpose of the description of certain embodiments. These figures may not precisely reflect the characteristics of any given embodiment, and are not necessarily intended to define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

As discussed above, the present technology provides equipment, systems and methods for growing aquatic organisms. In various embodiments, the present technology is used for cultivating (growing) algae. However, other aquatic organisms may be cultivated, including aqueous plant and animal species, using the same or similar equipment, systems and methods. In various embodiments, plant species may be characterized as phytoplankton or components thereof. In various embodiments, such animal species may be characterized as zooplankton or components thereof. For example, equipment, systems and methods of the present technology may be used in the cultivation of vertebrate or invertebrate animals, such as shrimp, mollusks, and sea cucumbers.

Currently algae may be cultivated on a commercial scale using pond systems, bubble column, horizontal pipes, flat panel, etc., methods. Pond systems are the most common system of algae cultivation and use shallow (typically one-foot deep) ponds, from about 1 acre to several acres in size, in which the algae are exposed to natural solar radiation (sunlight) which they convert into biomass. Bubble column systems are often used for cultivation of green microalgae and utilize a photo bioreactor system where algae inoculum originally grown in bottles is concentrated and transferred to photobioreactors for cultivation in batches. Horizontal pipe systems are similar to bubble column systems in that they provide a photo bioreactor for cultivation of algae. Flat-panel systems provide flat panel photobioreactors and are often ideally suited in wastewater treatment scenarios.

Cultivation of algae on a commercial scale has been fraught with many challenges due to the complexities that each algae strain possesses. Open pond systems have been the most productive as they can somewhat simulate the natural parameters algae depend on for growth, but they still have limitations. Other systems such as bubble column, horizontal pipes, flat panel, etc., also have positive and negative effects on the algae. All of these systems try to force growth instead of allowing the algae to propagate naturally.

Without limiting the scope, mechanism, function or utility of the present technology, the gliding wave reactors of the present technology may be based on nature's ability to create favorable conditions for cultivating algae, such as in a “biomimicry” modeling of natural systems. In various embodiments, biomimicry, which may afford benefits in the cultivation of algae, is utilized in optimizing the design of the gliding wave reactors since nature has optimized systems and continues to optimize itself. For example, the equipment, systems and methods of the present technology are operable to disperse nutrient (e.g., though upwelling) and exposing the aquatic plants or animals to food, and assist in the disruption of the boundary layer which is crucial for nutrient uptake.

Each algae strain has unique needs and guidelines for growth, because each algae strain is unique, with fairly specific needs for growth, often balancing multiple environmental factors (e.g., water movement, agitation, light, and temperature), which may be referred to as a “Goldilocks” environment. Thus, there is a need, as provided by the present technology in various embodiments, to provide a system for growing algae that can be adapted to optimize growth of each of a variety of algae species, while offering commercial benefits such as ease of use, efficiency in use of space, ease of assembly, and portability.

In the art, a “one method” reactor, such as an open pond, may be limited and cannot accommodate the various unique needs and guidelines for growth of different algae strains. For example, a Goldilocks environment is demanded by algae as conditions have to be just right to initiate quorum sensing where cells share information by chemical signaling. This chemical signal tells algae when to divide and bloom, when to slow down and sleep, and many other parameters. An important aspect of algae communication is the cell to cell interaction. This signaling is greatly enhanced due to environmental conditions so an ideal habitat must be able to adapt and create these conditions. In various embodiments, the gliding wave reactors of the present technology utilizes biomimicry to create such environmental conditions.

One environmental condition necessary for an ideal habitat is motion. Most algae flourishes in still to gently moving waters; one will notice that algae is never found in a waterfall. However, algae may be found on the edges where there is less agitation. Motion creation is where open ponds do well. Open pond systems generally have a paddle that gently pushes water to create gently moving waters that are an ideal habitat. However, the motion is often varied because the paddle is fixed to one point, leading to harsher or faster motion near the paddle and decreasing motion with distance from the paddle. Additionally, the openness of the ponds used in the open pond systems are also ideal for contamination. Another cultivation method utilizing bubble columns operates in a closed environment, which is ideal for dealing with contamination; however, the bubbles used to move the water/algae in the bubble column system also destroy the algae (this is called the sparger effect). Pumping algae through pipes damages the algae, because the paddles or gears inside the pump housing wound the algae.

An exemplary algae strain, Haematococcus pluvialis, requires very gentle to no movement for cell dividing during the growth phase, while the stressing phase requires increased motion to expose the cells to light and stress. Previously used cultivation systems cannot adapt the respective environments to accommodate the Haematococcus pluvialis. However, the gliding wave reactor of the present technology utilizes wave motion as a kinetic energy approach to moving the water/algae and can create a Haematococcus pluvialis-specific environment. The movement of the water/algae in the gliding waver reactor of the present technology can go from a gentle glide to a robust rock dependent on what is required during the different stages of cultivation.

Another environmental condition necessary for an ideal habitat is nutrition. Algae exists in nature with a balancing function: blooming when food is readily available and existing in encystment (dormancy) during famine.

Conditions are ideal for algae reproduction when nutrients are released into the algae's environment through agricultural runoff, sewage disposal, and/or upwelling. Upwelling occurs when the nutrients have settled on the bottom and a motion pushes the nutrients up. Algae may then consume the nutrients along with sunlight and other parameters to induce an algae bloom.

Open ponds are effective at generating motion but settling occurs further down the motion path, so algae do not get the full impact of nutrient dispersal. Bubble columns help lift the nutrients from the bottom but, as stated before, the sparger effect limits the ability of algae to grow. Alternatively, the gliding wave reactors of the present technology generates wave motion that mimics the upwelling of nutrients, creating a uniform environment for all the algae to feast on the nutrients provided in a peaceful atmosphere.

An additional environmental condition necessary for an ideal habitat is light. While some algae can grow in the dark by feeding on sugars or other compounds, the discussion herein will relate to photosynthetic algae cultivation (however, it is understood that the gliding wave reactor may accommodate algae that can grow in the dark). Photosynthetic algae utilize dissolved inorganic CO2, the optimal conditions previously discussed, and absorbing solar energy to create many different compounds. This is where the Goldilocks scenario can be particularly significant. An excess of solar energy can cause photoinhibition and damage the algae or not allow for light-induced growth.

In nature, algae look for a balanced space having both nutrition and light. In commercial systems, the light is forced on the algae, leading to one of the main problems with scale up, since the energy balance is on the wasteful side. When too much light is thrusted on the algae it causes photoinhibition, but also an unnecessary waste of money as the excessive light turns into heat that introduces more issues. However, the gliding wave reactor of the present technology shines in this area, as the various mixing motions produced by the gliding wave reactor bring the algae to the light instead of forcing the light into the water. The wave motions of the gliding wave reactor also reduce the shading effect which further expose the algae to the light. These result in only a minimal amount of light being needed for the perfect growing environment, which leads to a reduction in cost for creating an ideal habitat.

Equipment & Systems

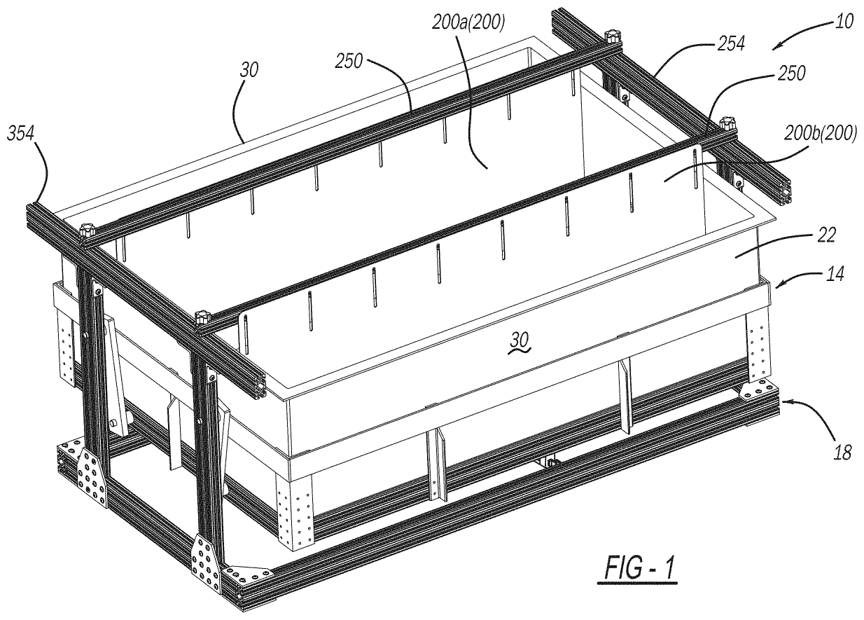

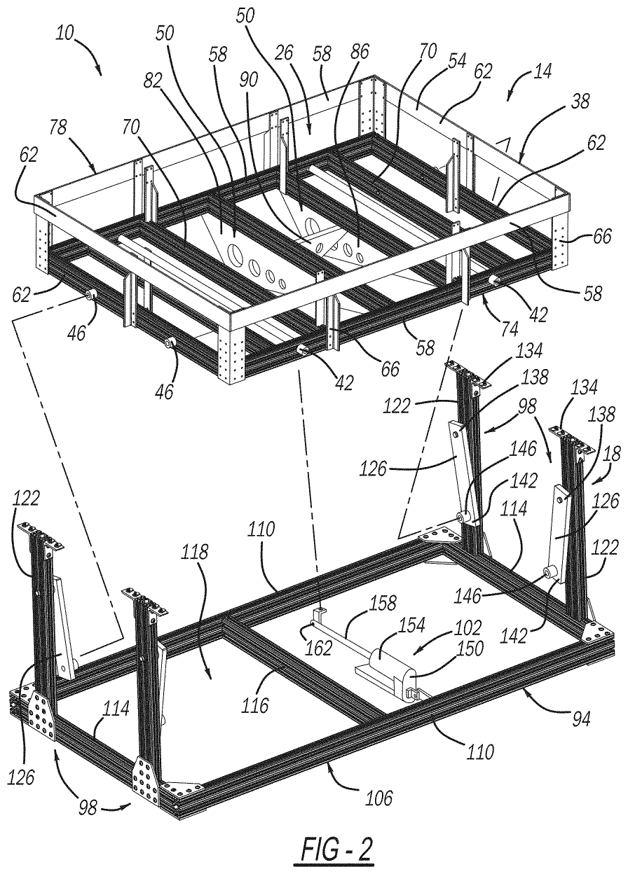

Referring to FIG. 1, an example embodiment of a gliding wave reactor 10 according to the present technology is illustrated. In at least one example embodiment the gliding wave reactor 10 includes a tank cradle 14, a base 18, and a tank 22. The base 18 may be stationary on the ground or other platform. The tank cradle 14 may be suspended on the base 18 such that the tank cradle 14 can make various movements in relation to the base 18. The tank 22 may be supported within a cavity 26 (FIG. 2) defined by the tank cradle 14.

The tank 22 may include sidewalls 30 and a base 34, but may not have a top, such that it functions as an open air container. In at least one example embodiment, the tank 22 may have a rectangular cross-section. While a rectangular cross-section is illustrated and described, it is understood that the tank 22 could have any cross-section (for example, circular, triangular, polygonal, etc.) as long as the cross-sectional shape of the tank 22 matches a cross-sectional shape of the tank cradle 14.

In at least one example embodiment, the tank 22 may be formed of a moldable polymer (such as polypropylene or plastic), a metallic material (e.g., aluminum, tin, stainless steel, or the like), a combination of materials, or any other suitable material or combination thereof.

The tank 22 may be configured to house an aquatic environment, such as an aquatic environment optimal for cultivating algae, as previously discussed. While cultivating algae is one use for the gliding wave reactor discussed herein, it is understood that this is not the only use. The gliding wave reactor may create optimal environments/habitats for cultivating and raising a variety of aquatic life, such as marine animals, small aquarium or ornamental fish, shrimp, baby tilapia, seaweed, or any other aquatic or marine plant or animal.

In at least one example embodiment, the tank 22 may house water, or another liquid, algae in various stages of growth, algae beads, an algae food source (for example, chemical nutrients, such as phosphorus and nitrogen). The tank 22 may be a 500 liter (L) or 1000 L tank, meaning that the tank 22 may be sized to house 500 L or 1000 L of liquid. However, it is understood that the tanks 22 may be larger (even much larger) than 1000 L, for example to create environments for raising seaweed or fish. Since the tank 22 is suspended in the tank cradle 14, the tank cradle 14 could be modified to accommodate any size tank.

Referring to FIGS. 1 and 2, in at least one example embodiment, the tank cradle 14 may include a frame 38, a pair of side pivot rods 42, a pair of end pivot rods 46, and at least one supporting brace 50 (for example, 3 supporting braces, as illustrated in FIG. 2). In at least one example embodiment, the frame 38 may be a modular frame, including frame segments 54 that may be bolted, or otherwise fixed, together to form the frame 38. The frame segments 54 may include longitudinal frame segments 58, end frame segments 62, upright frame segments 66, and cross-bracing frame segments 70. Each of the individual frame segments 54 is sized such that the frame segments 54, when disassembled, can all fit within the tank 22. With all parts fitting within the tank 22, shipment may be easy, cost-effective, and efficient.

The longitudinal frame segments 58 and end frame segments 62 may be assembled to form two rectangles, a lower rectangle 74 and an upper rectangle 78, that are spaced vertically and connected by the upright frame segments 66. For example, the longitudinal frame segments 58 may extend a length of the frame 38 and may be connected on the ends by the end frame segments 62. The longitudinal frame segments 58 of one of the two rectangles (for example lower rectangle 74) may be connected to the longitudinal frame segments 58 of the other of the two rectangles (for example upper rectangle 78) by the upright frame segments 66. The cross-bracing frame segments 70 may extend orthogonally, from one longitudinal frame segment 58 on the lower rectangle 74 to another longitudinal frame segment 58 on an opposing side of the lower rectangle 74 to provide support for the tank 22 in the tank cradle 14.

The frame 38, when assembled, may be a rectangular frame that defines the cavity 26 therein. Although the frame 38 is illustrated and described as having a rectangular cross-section, it is understood that the frame 38 could have any cross-section (for example, circular, triangular, polygonal, etc.) as long as the cross-sectional shape of the frame 38 matches the cross-sectional shape of the tank 22.

In at least one example embodiment, the frame 38, or frame segments 54, may be formed of a moldable polymer (such as polypropylene or plastic), a metallic material (e.g., aluminum, tin, stainless steel, or the like), a combination of materials, or any other suitable material or combination thereof.

The side pivot rods 42 may be elongated cylindrical rods extending parallel to the cross-bracing frame segments 70. The side pivot rods 42 may extend orthogonally to the longitudinal frame segments 58, from one longitudinal frame segment 58 on the lower rectangle 74 to another longitudinal frame segment 58 on an opposing side of the lower rectangle 74. The side pivot rods 42 may project through and outwardly from the longitudinal frame segments 58 on each side of the lower rectangle 74, providing pivot rods for attaching and suspending the tank cradle 14 from the base 18.

The end pivot rods 46 may be cylindrical rods extending orthogonally to the end frame segments 62 on the lower rectangle 74. The end pivot rods 46 may project outwardly from the end frame segments 62 on each end of the lower rectangle 74, providing pivot rods for attaching and suspending the tank cradle 14 from the base 18.

In at least one example embodiment, the side pivot rods 42 and end pivot rods 46 may be formed of a moldable polymer (such as polypropylene or plastic), a metallic material (e.g., aluminum, tin, stainless steel, or the like), a combination of materials, or any other suitable material or combination thereof.

The supporting braces 50 may be triangularly-shaped braces that are positioned to provide stability and support to the tank cradle 14. While the supporting braces 50 are illustrated and described as being triangular-shaped braces, it is understood that the supporting braces 50 could be any shape (for example, circular, triangular, polygonal, etc.).

In at least one example embodiment, the supporting braces 50 may include a first supporting brace 82, a second supporting brace 86, and a third supporting brace 90. The first supporting brace 82 may be fixed to a bottom side of one of the cross-bracing frame segments 70 on a long side, or hypotenuse, of the first supporting brace 82. The second supporting brace 86 may be fixed to a bottom side of another of the cross-bracing frame segments 70 on a long side, or hypotenuse, of the second supporting brace 86. The third supporting brace 90 may be positioned orthogonally to, and connecting, the first supporting brace 82 and the second supporting brace 86. The third supporting brace 90 may be connected or fixed to the bottom sides of the cross-bracing frame segments 70, the first supporting brace 82 and the second supporting brace 86, or both.

In at least one example embodiment, the supporting braces 50 may be formed of a moldable polymer (such as polypropylene or plastic), a metallic material (e.g., aluminum, tin, stainless steel, or the like), a combination of materials, or any other suitable material or combination thereof. In at least one example embodiment, the supporting braces 50 may include one or more apertures for weight reduction.

With continued reference to FIGS. 1 and 2, the base 18 may include a frame 94, suspension brackets 98, and an actuator 102. In at least one example embodiment, the frame 94 may be a modular frame, including frame segments 106 that may be bolted, or otherwise fixed, together to form the frame 94. The frame segments 106 may include longitudinal frame segments 110 and end frame segments 114. Each of the individual frame segments 106 is sized such that the frame segments 106, when disassembled, can all fit within the tank 22. With all parts fitting within the tank 22, shipment may be easy, cost-effective, and efficient.

The longitudinal frame segments 110 and end frame segments 114 may be assembled to form a rectangle. For example, the longitudinal frame segments 110 may extend a length of the frame 94 and may be connected on the ends by the end frame segments 114. In an example embodiment, the frame 94 could additionally include one or more cross-bracing frame segments 116 that extend orthogonally, from one longitudinal frame segment 110 to another longitudinal frame segment 110 on an opposing side to provide additional support.

The frame 94, when assembled, may be a rectangular frame that defines a cavity 118 therein. Although the frame 94 is illustrated and described as being a rectangular frame, it is understood that the frame 94 could have any shape (for example, circular, triangular, polygonal, etc.).

In at least one example embodiment, the frame 94, or frame segments 106, may be formed of a moldable polymer (such as polypropylene or plastic), a metallic material (e.g., aluminum, tin, stainless steel, or the like), a combination of materials, or any other suitable material or combination thereof.

In at least one example embodiment, the suspension bracket 98 may include a vertical post 122 and a swing arm 126. The vertical post 122 may be fixed, such as bolted, to the frame 94 on a first end 130. The swing arm 126 may be pivotably, or rotatably attached to a second end 134 of the vertical post 122. For example, a first end 138 of the swing arm 126 may be attached to the second end 134 of the vertical post 122 by a pin, bearing, or other fixture that allows the swing arm 126 to rotate relative to the vertical post 122. A second end 142 of the swing arm 126 may be pivotably, or rotatably, attached to one of the side pivot rods 42 or end pivot rods 46 of the tank cradle 14 (FIG. 1) such that the swing arm 126 may rotate or pivot relative to the tank cradle 14. For example, the second end 142 may include an aperture housing a bearing 146 that receives one of the side pivot rods 42 or end pivot rods 46 therein.

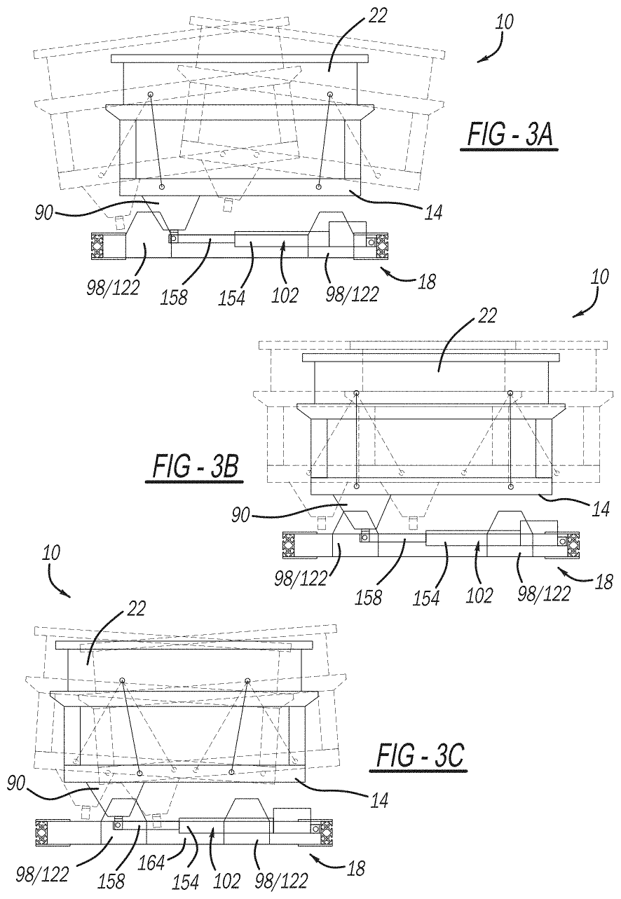

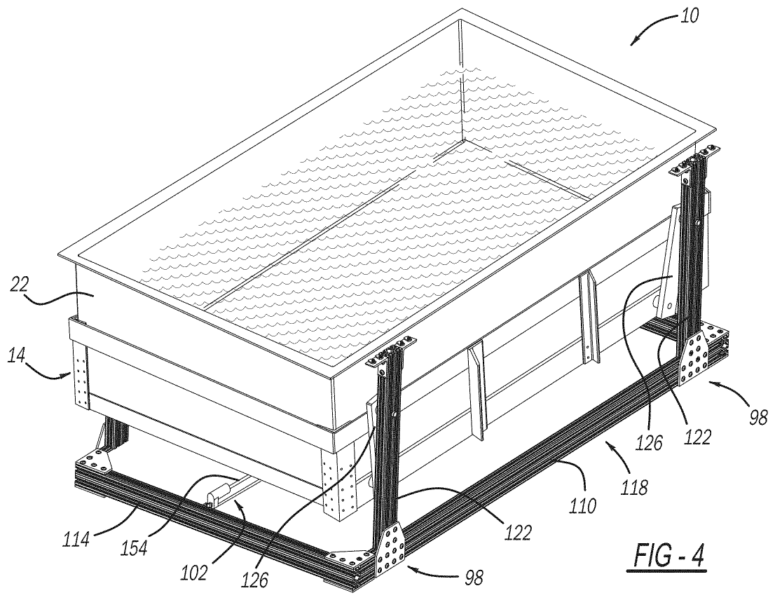

In at least one example embodiment, the base 18 may include multiple suspension brackets 98. For example, four suspension brackets 98 may be fixed to the frame 94 to suspend the tank cradle 14. In at least one example embodiment, if the tank cradle 14 is to be suspended on its sides (FIG. 4), two suspension brackets 98 are fixed to each of the longitudinal frame segments 110 of the frame 94 of the base 18. If the tank cradle is to be suspended on its ends (FIG. 1), suspension brackets 98 are fixed to each of the end frame segments 114 of the frame 94 of the base 18. The placement of the vertical posts 122 along either the longitudinal frame segment 110 or the end frame segment 114 may influence a motion of the tank cradle 14, tank 22, and/or contents of the tank 22 (further described with respect to FIGS. 3A-3C, below).

In at least one example embodiment, the actuator 102 may be a linear actuator having a servo motor 150, a housing 154, and a rod 158. The rod 158 may be slidable into and out of an aperture in the housing 154. The servo motor 150 may be attached to the rod 158 and mounted with the housing 154, such that the servo motor 150 controls movement of the rod 158 into and out of the aperture in the housing 154. In at least one example embodiment, the servo motor 150 may be a 48 volt (V) and a 1-100 mm linear actuator motor.

In at least one example embodiment, the housing 154 of the actuator 102 may be fixed to either the longitudinal frame segment 110 or the end frame segment 114 of the frame 94 of the base 18. For example, if the tank cradle 14 is to be suspended on its sides (FIG. 4), the actuator 102 is fixed to one end frame segment 114 of the frame 94 of the base 18. If the tank cradle is to be suspended on its ends (FIG. 1), the actuator is fixed to one longitudinal frame segment 110 of the frame 94 of the base 18.

In at least one example embodiment, a free end 162 of the rod 158 may be fixed to the supporting brace 50 of the tank cradle 14. For example, the rod 158 may be pivotably fixed to the third support brace 90 by a pin or other fastener. As further described below and depending on the placement of the suspension brackets 98, when the rod 158 is driven out of the housing 154 by the motor 150, the tank cradle 14 is pushed away from the housing 154 and tilted, or rocked, toward the housing 154 (i.e., clockwise), and when the rod 158 is pulled into the housing 154 by the motor 150, the tank cradle 14 is pulled toward the housing 154 and tilted, or rocked, away from the housing 154 (i.e., counterclockwise).

Now referring to FIGS. 1 and 3A-3C, during use, the gliding wave reactor 10 may have customized movements to optimize the environment in the tank 22. With an optimized environment, algae outcompete any component that could contaminate the cultivation. Further, the optimized environment may change according to the life cycle stage of the algae. For example, in a specific type of algae, Haematococcus pluvialis, the algae grows in one environment and then if it is stressed in a different environment, the Haematococcus pluvialis produces a compound that is a powerful antioxidant (used for sunblock) and provides an energy source (i.e. food) for other aquatic life (the substance that makes salmon and shrimp pink, for example). With the gliding wave reactor 10 disclosed herein, the environments may be changed, and optimized, to support this type of algae.

Referring to FIG. 3A, the vertical posts 122 of the suspension brackets 98 are fixed on opposing ends of the end frame segment 114 of the base 18. As the rod 158 of the actuator 102 moves in and out of the housing 154, the tank 22 is moved in an inverse rock motion. Thus, when the rod 158 is fully extended away from the housing 154, the tank 22 rocks away from the outside vertical post 122, in a counter clockwise direction, and when the rod 158 is fully inserted within the housing 154, the tank 22 rocks away from the inside vertical post 122, in a clockwise direction. Thus, the inverse rock motion ends up looking like a hill or an arc.

Referring to FIG. 3B, the vertical posts 122 of the suspension brackets 98 are fixed slightly spaced from opposing ends of the end frame segment 114 of the base 18. The vertical posts 122 here, are closer together than the vertical posts 122 pictured in FIG. 3A. As the rod 158 of the actuator 102 moves in and out of the housing 154, the tank 22 is moved in a gliding motion. Thus, when the rod 158 is fully extended away from the housing 154, the tank 22 glides away from the outside vertical post 122, but remains relatively flat and level, and when the rod 158 is fully inserted within the housing 154, the tank 22 glides away from the inside vertical post 122, but remains relatively flat and level. Thus, the glide motion ends up looking like horizontal line running parallel to the end frame segment 114 of the base 18.

Referring to FIG. 3C, the vertical posts 122 of the suspension brackets 98 are fixed spaced from opposing ends of the end frame segment 114 of the base 18 and close together. For example only, the vertical posts 122 of the suspension brackets 98 are fixed closer to a center 164 of the end frame segment 114 than the end of the end frame segment 114. The vertical posts 122 here, are closer together than the vertical posts 122 pictured in FIG. 3B. As the rod 158 of the actuator 102 moves in and out of the housing 154, the tank 22 is moved in a rocking motion. Thus, when the rod 158 is fully extended away from the housing 154, the tank 22 rocks upward and away from the outside vertical post 122, in a clockwise direction, and when the rod 158 is fully inserted within the housing 154, the tank 22 rocks upward and away from the inside vertical post 122, in a counter clockwise direction. Thus, the rocking motion ends up having a U-shape or looking like a rocking baby's crib.

Now referring to FIG. 4, the gliding wave reactor 10 includes the pieces/parts discussed herein that may all be bolted/unbolted/adjusted easily, allowing for different configurations for the gliding wave reactor 10. In at least one example embodiment, the suspension brackets 98 may be fixed on the longitudinal frame segments 110 of the base 18. Additionally, the actuator 102 may be fixed on the end frame segment 114 of the base 18. Suspending the tank cradle 14 and the tank 22 along the longitudinal sides changes the flow of the liquid in the tank 22 (as compared to when the tank cradle 14 and tank 22 are suspended from the ends) when the actuator 102 is activated.

Similarly as described with reference to FIGS. 3A-3C, with the side-suspended configuration in FIG. 4, the gliding wave reactor 10 may have customized movements to optimize the environment in the tank 22. With an optimized environment, algae outcompete any component that could contaminate the cultivation. When the vertical posts 122 of the suspension brackets 98 are fixed on opposing ends of the longitudinal frame segment 110 of the base 18, the tank 22 is moved in the inverse rock motion as the rod 158 of the actuator 102 moves in and out of the housing 154. When the vertical posts 122 of the suspension brackets 98 are fixed slightly spaced from opposing ends of the longitudinal frame segment 110 of the base 18 (i.e. closer together than the vertical posts 122 in the inverse rock motion), the tank 22 is moved in a gliding motion as the rod 158 of the actuator 102 moves in and out of the housing 154. When the vertical posts 122 of the suspension brackets 98 are fixed such that the suspension brackets 98 are spaced from opposing ends of the longitudinal frame segment 110 of the base 18 and closer to a center 168 of the longitudinal frame segment 110 than an end of the longitudinal frame segment 110 (i.e., closer together than the vertical posts 122 during the gliding motion), the tank 22 is moved in a rocking motion as the rod 158 of the actuator 102 moves in and out of the housing 154.

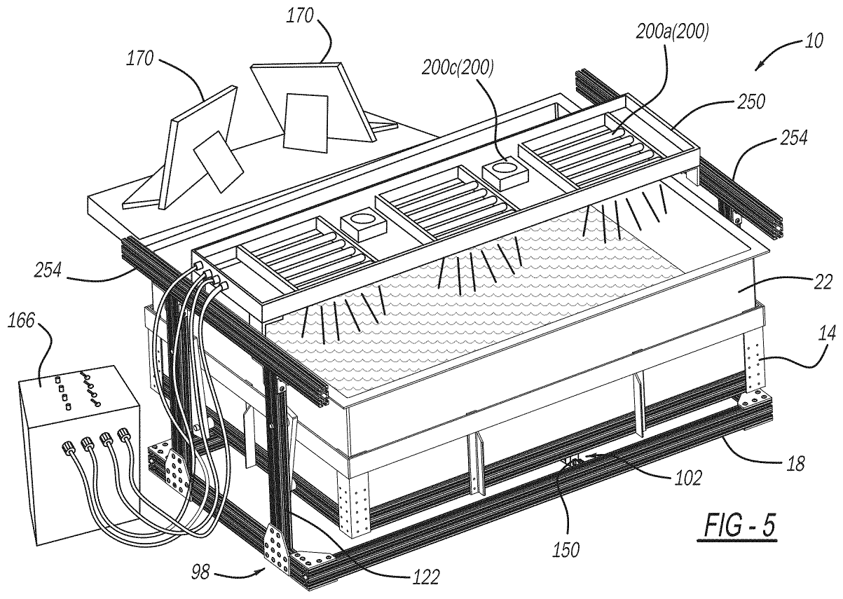

Now referring to FIG. 5, in at least one example embodiment, the gliding wave reactor 10 may be connected to a battery 166. For example only, the actuator motor 150 may be connected to the battery 166 and may receive power to operate and move the rod 158.

In at least one example embodiment, the gliding wave reactor 10 may be in communication with (either wired or wireless) at least one controller 170. As illustrated, the controller 170 may be a remote controller housed in a computer. Alternatively, the controller 170 could be attached to or housed within the gliding wave reactor 10. The controller 170 may utilize software to control actuation of the motor 150 on the actuator 102, controlling the movement of the tank 22.

In at least one example embodiment, the gliding wave reactor 10 may additionally support various attachments 200, such as light and/or heat lamps 200a, partitions 200b (FIG. 1) for stirring or interrupting the water current, cameras 200c, sensors, automatic feeders, or any other type of accessory or attachments used in the cultivation of marine life. The various attachments 200 may be fixed to longitudinally extending arms 250 that are fastened (such as by bolting, etc.) to horizontally extending beams 254 on both ends and supported by the suspension brackets 98. For example, the horizontally extending beams 254 may be bolted, or otherwise removably fixed to the vertical posts 122 of the suspension brackets 98.

The various attachments 200 may be connected to the battery 166 to receive power for operation and may be in communication with (either wired or wireless) controller 170. When the controller 170 is housed in the computer, the computer may serve as a display for providing a visual image of the data from the various attachments 200 and/or may utilize software to control actuation of the various attachments 200.

In use, the gliding wave reactor 10 is shipped to a customer in a reasonably flat container with all parts stored within the tank 22. The shipping configuration and modular assembly of the gliding wave reactor 10 is much cheaper than currently available options, making the gliding wave reactor 10 a possible option for every day, individual consumers instead of only commercial operations. Upon opening the box, the tank 22 and all components may be removed.

The various parts are easily assembled into the tank cradle 14 and base 18, requiring only basic tools to bolt together. The modular assembly is also easily reconfigured to meet the specific needs of the various types of marine life that can be cultivated in the tank 22. For example, the suspension brackets 98 may be moved to support suspension on the ends of the tank 22 or to support suspension on the sides of the tank 22. Additionally, the suspension brackets 98 may be customized at different widths along the longitudinal frame segment 110 and the end frame segment 114 to achieve the various motions (i.e., inverse rock, gliding, and rocking). Thus, the gliding wave reactor 10 is fully customizable to achieve the optimum environment for the specific needs of the various types of marine life that can be cultivated in the tank 22, even the most delicate of algae, such as the Haematococcus pluvialis.

In at least one example embodiment, the linear actuator motor 150 may include sensors that sense where the motor 150 hits a stall and releases the rod 158 to return. Alternatively, the controller 170 may utilize software that communicates both with various attachments 200 (such as sensors) and the motor 150 to determine an optimum time for releasing the rod 158. Thus, the gliding wave reactor 10 may optimize use of the actuator 102 to achieve maximum results. Additionally, the gliding wave reactor 10 may be energy efficient in that the controller 170 may control the actuator 102 to ride with the wave/motion of the water (instead of working against it), such that movement of the tank 22 requires less energy.

### Methods

The present technology provides methods for cultivating algae and other aquatic organisms using wave reactors. In various embodiments, methods comprise introducing an aqueous media to the wave reactor, introducing an aquatic organism to the media, and incubating the organism.

Aqueous media useful herein include water and aqueous compositions that may comprise nutrients and other materials that aid in one or more of the growth of the organisms to be cultivated, processing of the organisms, or other benefits. Such compositions may be solutions or suspensions. In various embodiments, a dispersion of a water-insoluble suspension is maintained by agitation of the wave reactor by operation of the reactor as discussed above.

In some embodiments, the media comprise beads. For example, beads may comprise a microorganism such as unicellular microalgae, an insoluble carbon source such as char, water, and a crosslinked organic matrix. The beads can further contain clay, such as kaolin.

The crosslinked organic matrix may comprise polymers, such that the organic matrix before crosslinking contains a plurality of hydroxyl groups that react with a divalent cation to crosslink the matrix. Suitable polymers used in various embodiments of the current teachings include water soluble polysaccharides, such as sodium alginate, the class of galactomannans, gellan gum, carrageenan, and agarose. Divalent cations of the alkaline earth metals may be used to crosslink the organic matrix to form the beads. In a particular embodiment, the crosslinking cation is provided in the form of calcium chloride.

In various embodiments, the insoluble carbon source is a char such as a biochar. Biochar may be produced from a suitable biomass, such as algae, rice hulls, and other forms of biomass. The beads preferably further contain cations and anions corresponding to the nutrients required to grow a strain of algae before it is incorporated into the algal beads of the current technology.

In various embodiments, beads are solid or semi-solid pellets comprising algae and a polymer matrix. Without limitation, the beads may have a diameter of from about 1 mm to about 2 cm in diameter, or from about 2 mm to about 9 mm, or from about 3 mm to about 6 mm in diameter. The beads may have any three-dimensional shape, but in many embodiments are spheroidal. As referred to herein a “spheroidal” pellet or bead may be truly spherical, essentially spherical, or spheroidal, and may have irregular features (such as described below regarding flat, convex or concave surface features). In some embodiments, the beads are polyhedral, such as resembling an icosahedron. The surface may be smooth (including substantially smooth surfaces having minor irregularities), rough, or having ribs or other repeating or random surface structures. Without limiting the scope, mechanism, function or utility of present technology, it is believed that beads having such irregular surface features facilitate greater mixing of beads in a bioreactor vessel relative to spheroidal beads, by one or more of altering their motion through culture media, disrupting the boundary layer effect of liquid media on the algae, and reducing adherence of algae to surfaces within a bioreactor. For example, beads having irregular features may spin as they travel through media or bounce in a random fashion as they impact the walls of a bioreactor vessel.

Beads among those useful herein are described in U.S. Pat. No. 9,243,219, Dimitrelos, issued Jan. 26, 2016, and U.S. Patent Application Publication 2019/0225931, Dimitrelos, published Jul. 25, 2019, both incorporated by reference herein.

As discussed above, the equipment, systems and methods of the present technology may be used for cultivating algae, in particular microalgae. The term microalgae embraces species described as cyanobacteria, as well as green, red, and brown algae. Microalgae among those useful herein include Eugelenophyta, Chyrisphyta, Pyrrophyta, Chlorophyta, Rhodophyta, and Xanaophyta. Genera of algae useful herein include Aphanizomenon, Arthrospira, Chlorella, Crypthecodinium, Dunaliella, Euglena, Haematococcus, Isochrysis, Nannochloropsis, Odontella, Phaeodactylum, Picochlorum, Porphyridium, Scenedesmus, Schizochytrium, Spirulina, and Tetraselmis.

In various embodiments, the compositions and methods of the present technology employ phototropic production of Haematococcus, Nannochloropsis, Picochlorum, Chlorella, Spirulina, and Dunaliella. In various embodiments, compositions and methods employ heterotrophic or mixotrophic production of Euglena. In particular, in various embodiments, the compositions and methods of the present technology employ algae selected from the group consisting of Haematococcus species (e.g. H. pluvialis), Chlorella species (e.g. C. vulgaris, C. zofringiensis), Chaetoceros species., Dunaliella salina, Phaeodactylum tricornutum, Porphyridium cruentum, Rhodella species, Skeletonema species, Scenedesmus species, and spirulina (e.g. Arthrospira maxima and Arthrospira platensis).

In various embodiments, Haematococcus are among the commercially important microalgae. For example, Haematococcus pluvialis is the richest natural source of astaxanthin which is considered to be a “super anti-oxidant.” Natural astaxanthin produced by H. pluvialis has significantly greater antioxidant capacity than synthetic astaxanthin. Astaxanthin has important applications in the nutraceuticals, cosmetics, food, and aquaculture industries.

### Non-limiting Discussion of Terminology

The headings (such as “Introduction” and “Summary”) and sub-headings used herein are intended only for general organization of topics within the present technology, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Numerous specific details are set forth such as examples of specific components, devices, and methods, to provide a thorough understanding of embodiments of the present technology. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. Equivalent changes, modifications and variations of specific embodiments, devices, components, materials, compositions and methods may be made within the scope of the present technology, with substantially similar results. Moreover, recitation of multiple embodiments having stated features is not intended to exclude other embodiments having additional features, or other embodiments incorporating different combinations of the stated features.

For example, a component which may be A, B, C, D or E, or combinations thereof, may also be defined, in some embodiments, to be A, B, C, or combinations thereof. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and combinations thereof. Similarly, the terms “can” and “may” and their variants are intended to be non-limiting, such that recitation that an embodiment can or may comprise certain elements or features does not exclude other embodiments of the present technology that do not contain those elements or features.

The method steps, processes, and operations described herein are not to be construed as necessarily requiring their performance in the particular order discussed or illustrated, unless specifically identified as an order of performance. It is also to be understood that additional or alternative steps may be employed.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

“A” and “an” as used herein indicate “at least one” of the item is present; a plurality of such items may be present, when possible.

Numeric values stated herein should be understood to be approximate, and interpreted to be about the stated value, whether or not the value is modified using the word “about.” Thus, for example, a statement that a parameter may have value “of X” should be interpreted to mean that the parameter may have a value of “about X.” “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Further, the phrase “from about A to about B” includes variations in the values of A and B, which may be slightly less than A and slightly greater than B; the phrase may be read be “about A, from A to B, and about B.” Disclosure of values and ranges of values for specific parameters (such as temperatures, molecular weights, weight percentages, etc.) are not exclusive of other values and ranges of values useful herein.

It is also envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9,1-8,1-3,1-2,2-10,2-8,2-3, 3-10, and 3-9.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A gliding wave reactor comprising:
a base;
a tank configured to hold a liquid;
a suspension bracket configured to suspend the tank over the base; and
an actuator fixed to the base and configured to move the tank to create an environment in the liquid for cultivating marine life.

2. The gliding wave reactor of claim 1, further comprising a tank cradle being attached to the suspension bracket and defining a cavity for supporting the tank.

3. The gliding wave reactor of claim 2, wherein the suspension bracket includes a vertical post and a swing arm, the swing arm being rotatably attached to the vertical post, the vertical post being fixed to the base, and the swing arm being rotatably attached to the tank cradle.

4. The gliding wave reactor of claim 2, wherein the base and the tank cradle are modular assemblies.

5. The gliding wave reactor of claim 1, wherein the suspension bracket is one of a pair of suspension brackets, the pair of suspension brackets being disposed on either an end of the base or a side of the base for suspending the tank on either an end of the tank or a side of the tank, respectively.

6. The gliding wave reactor of claim 5, wherein when the pair of suspension brackets are positioned on opposing ends of the end of the base or the side of the base, the actuator moves the tank in an inverse rocking motion.

7. The gliding wave reactor of claim 5, wherein when the pair of suspension brackets are positioned spaced from opposing ends of the end of the base or the side of the base, the actuator moves the tank in a gliding motion.

8. The gliding wave reactor of claim 5, wherein when each of the pair of suspension brackets are positioned closer to a center of the end of the base or the side of the base than an end of the end of the base or the side of the base, the actuator moves the tank in a rocking motion.

9. The gliding wave reactor of claim 1, wherein the actuator is a linear actuator including a motor, a rod, and a housing.

10. The gliding wave reactor of claim 9, wherein the rod of the actuator is fixed to a single point on the tank.

11. The gliding wave reactor of claim 1, further comprising an attachment fixed to a longitudinal arm supported by the suspension bracket and extending over an open top of the tank.

12. The gliding wave reactor of claim 11, wherein the attachment is a light, a heat lamp, a partition, a camera, a sensor, an automatic feeder, or a combination thereof.

13. The gliding wave reactor of claim 1, further comprising a controller controlling the actuator.

14. An algae-cultivating apparatus comprising:
a tank configured to hold a liquid;
a suspension bracket configured to suspend the tank;
an actuator fixed to the tank and configured to move the tank to create an environment in the liquid for cultivating algae; and
a controller configured to control actuation of the actuator and movement of the tank.

15. The algae-cultivating apparatus of claim 14, further comprising a base, the suspension bracket being fixed to the base.

16. The algae-cultivating apparatus of claim 15, further comprising a tank cradle being attached to the suspension bracket and housing the tank.

17. The algae-cultivating apparatus of claim 16, wherein the suspension bracket includes a vertical post and a swing arm, the swing arm being rotatably attached to the vertical post, the vertical post being fixed to the base, and the swing arm being rotatably attached to the tank cradle.

18. The algae-cultivating apparatus of claim 16, wherein the base and the tank cradle are modular assemblies.

19. The algae-cultivating apparatus of claim 14, wherein the suspension bracket is one of a pair of suspension brackets, the pair of suspension brackets suspending the tank on either an end of the tank or a side of the tank.

20. The algae-cultivating apparatus of claim 14, wherein the actuator is a linear actuator including a motor, a rod, and a housing.
